(19) United States
(12) Patent Application Publication
(10) Pub. No.: US 2024/0188857 A1
(43) Pub. Date: Jun. 13, 2024

(54) BIOMEDICAL DETECTION PHOTOELECTRIC MODULE AND MANUFACTURING METHOD THEREOF

(71) Applicant: Taiwan-Asia Semiconductor Corporation, Hsinchu City (TW)

(72) Inventors: Sheng-Wei CHEN, Hsinchu City (TW); Chung-Cheng LIN, Hsinchu City (TW); Chuan-Fa LIN, Hsinchu City (TW); Chuan-Yu HUNG, Hsinchu City (TW)

(21) Appl. No.: 18/532,316

(22) Filed: Dec. 7, 2023

(30) Foreign Application Priority Data
Dec. 12, 2022 (TW) 111147676

(51) Int. Cl. A61B 5/1455; H01L 31/0203; H01L 31/0232; H01L 31/173; H01L 31/18

(52) U.S. Cl. CPC A61B 5/1455; H01L 31/0203; H01L 31/02325; H01L 31/173; H01L 31/18; A61B 2562/12

(57) ABSTRACT

The invention provides a biomedical detection photoelectric module, which includes a circuit substrate, a plurality of isolating barriers, a light-emitting unit, a photosensitive unit, and a light-guiding assembly. The light-guiding assembly provides a first optical element and a second optical element, respectively, corresponding to a light-emitting unit and a light-sensing unit. The first optical element has a first pattern layer to alter the emitting angle of the first light generated by the light-emitting unit. The second optical element has a second pattern layer to alter the incident angle of a second optical element entering the light-sensing unit. The first light is guided by the first optical element so that the energy of the first light is concentrated and incident on a predetermined area of a human's skin layer. The second light in the predetermined area is guided by the second optical element to enter the light-sensing unit. The present invention further provides a process method for the biomedical detection photoelectric module.

### CROSS-REFERENCES TO RELATED APPLICATIONS

This application claims the benefit of priority to Taiwanese Patent Application No. 111147676 filed on Dec. 12, 2022, which is hereby incorporated by reference in its entirety.

### BACKGROUND OF THE INVENTION

### Field of the Invention

The present invention relates to a biomedical detection component. More particularly, the present invention relates to a biomedical detection photoelectric module.

### Descriptions of the Related Art

Conventional biomedical detection components are manufactured by molding plastic PLC casings to form the light-emitting region and light-receiving region. After forming the light-emitting and light-receiving regions, processes such as die bonding, wire bonding, dispensing, encapsulation, and baking are employed to create the sensing module.

However, the isolation barrier (used to separate the light-emitting region from the light-receiving region) formed by molding the casing is made by the processes of molding or injection molding, resulting in a relatively large width>0.5 mm). Additionally, the encapsulation material is formed through dispensing approaches, resulting in uneven surfaces where optical coatings cannot be formed to resist noise. Furthermore, the light source located in the light-emitting region emits non-directed light, leading to excessive energy wastage.

In view of these issues, the present invention provides a biomedical detection photoelectric module and its manufacturing process to address the shortcomings of the prior art.

### SUMMARY OF THE INVENTION

An objective of the present invention is to provide a biomedical detection photoelectric module capable of measuring superficial skin conditions, such as the state of tissue fluid in subcutaneous tissue under the epidermal layer, including but not limited to measurements of blood glucose, blood oxygen, hemoglobin, bilirubin, etc.

One objective of the present invention is to provide a first optical element and a second optical element, each having a transmitting layer and a patterned layer, where the patterned layer not only guides light outwards in a specific direction (or inwards) but also enhances the efficiency of light emission and collection, based on the biomedical detection photoelectric module disclosed herein.

One objective of the present invention is to provide a first optical element and a second optical element that are flat (or planar) and can directly contact the surface of the skin to reduce the optical path for emitted and incident light, thereby achieving the objective of measuring superficial skin conditions, based on the biomedical detection photoelectric module disclosed herein.

One objective of the present invention is to form a plurality of chambers (e.g., two chambers or three chambers), preventing the light-emitting unit and the photosensitive unit in the chambers from interfering with each other, based on the biomedical detection photoelectric module disclosed herein.

To achieve the object described above, the present invention provides a biomedical detection photoelectric module for detecting a state of tissue liquid beneath the epidermis. The biomedical detection photoelectric module includes a circuit substrate, a plurality of isolating barriers, a light-emitting unit, a photosensitive unit, and a light-guiding assembly. The isolating barriers are formed on the circuit substrate. The isolating barriers form a first chamber and a second chamber on the circuit substrate. The first chamber is located on a first side of the second chamber. The light-emitting unit is disposed on the circuit substrate and situated in the first chamber. The light-emitting unit is capable of emitting a first light. The photosensitive unit is disposed on the circuit substrate and situated in the second chamber. The photosensitive unit is configured to receive a second light from the epidermis, in which the second light is diffusely reflected light of the first light. The light-guiding assembly is disposed between the isolating barriers to enclose the first chamber and the second chamber. The light-guiding assembly comprises a first optical element and a second optical element. The first optical element has a first transmitting layer formed on one side thereof and a first patterned layer formed on another side thereof. The second optical element has a second transmitting layer formed on one side thereof and a second patterned layer formed on the other side thereof. The first optical element corresponds to the light-emitting unit, and the first patterned layer alters an emitting angle of the first light, and the second optical element is corresponding to the photosensitive unit, and the second patterned layer alters an incident angle of the second light. The first light is guided and concentrated onto a predetermined region of the epidermis by the first optical element, and the second light from the predetermined region is guided and concentrated onto the photosensitive unit by the second optical element, in which the first light and the second light are blocked by the isolating barriers, preventing the direct incidence of the first light on the photosensitive unit.

One objective of the present invention is to provide a method for manufacturing a biomedical detection photoelectric module, in which the isolating barriers are formed by combining a semi-cutting process and a dispensing process (e.g., with a width less than or equal to 0.3 centimeters). This reduces the overall size of the biomedical detection photoelectric module, making it suitable for monitoring applications at short distances in superficial tissue

To achieve the object described above, the present invention provides a method for manufacturing a biomedical detection photoelectric module. The method comprises: step (a) forming a transmitting layer on one side of a quartz substrate and forming a first patterned layer and a second patterned layer on another side of the quartz substrate to form a light-guiding assembly having a first optical element and a second optical element; step (b) disposing a light-emitting unit and a photosensitive unit on a circuit substrate using a packaging process; step (c) forming a transparent glue layer on the circuit substrate to secure the light-emitting unit and the photosensitive unit on the circuit substrate; step (d) bonding the light-guiding assembly with the transparent glue layer on the circuit substrate; step (e) performing a semi-cutting process to cut the light-guiding assembly and the transparent glue layer to form at least one isolating groove; and step (f) injecting an opaque glue material into the isolating groove and curing the opaque glue material so to separate a first chamber from a second chamber.

### BRIEF DESCRIPTION OF THE DRAWINGS

Aspects of the present disclosure are best understood from the following detailed description when read with the accompanying Figures. It is noted that, in accordance with the standard practice in the industry, various features are not drawn to scale. In fact, the dimensions of the various features may be arbitrarily increased or reduced for clarity of discussion.

FIG. 1 is a cross-sectional view schematically depicting the structure of a biomedical detection photoelectric module according to the first embodiment of the present invention.

FIG. 2 is an enlarged view schematically illustrating the first patterned layer according to the first embodiment of the present invention.

FIG. 3 is an enlarged view schematically illustrating the second patterned layer according to the first embodiment of the present invention.

FIG. 4 is a cross-sectional view schematically depicting the structural of the biomedical detection photoelectric module according to the second embodiment of the present invention.

FIG. 5 is a flowchart illustrating a method for manufacturing a biomedical detection photoelectric module in accordance with the third embodiment of the present invention.

### DETAILED DESCRIPTION OF THE PREFERRED EMBODIMENT

The following disclosure provides many different embodiments, or examples, for implementing different features of the provided subject matter. Specific examples of components and arrangements are described below to simplify the present disclosure. These are, of course, merely examples and are not intended to be limiting.

In the present disclosure, the term “one” or “a” is used to describe the elements, components and assemblies mentioned herein. This is done for the sake of convenience and to provide a general understanding of the scope of the present invention. Therefore, unless expressly stated otherwise, such descriptions should be understood to comprise one or at least one, and the singular form also comprises the plural.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

The terms “comprising,” “having,” or any similar terminology are intended to encompass non-exclusive inclusions. For example, a component or structure with multiple elements is not limited to only the elements listed in this document but may also include other elements that are inherently typical for that component or structure even if not explicitly listed.

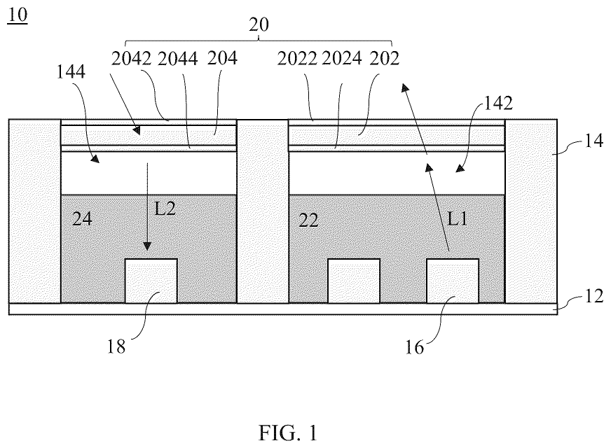

Refer to FIG. 1, which is a cross-sectional view schematically depicting the structure of a biomedical detection photoelectric module, according to the first embodiment of the present invention. In FIG. 1, the biomedical detection photoelectric module 10 is used for detecting the state of tissue fluid in subcutaneous tissue under the epidermal layer, such as blood glucose, blood oxygen, hemoglobin, bilirubin, etc. The biomedical detection photoelectric module can be applied in various fields such as telemedicine, home care, wearable devices, and more.

The biomedical detection photoelectric module 10 includes a circuit substrate 12′ a plurality of isolating barriers 14, a light-emitting unit 16, a photosensitive unit 18, and a light-guiding assembly 20.

The circuit substrate 12 has conductive wiring (not shown in the Figure) and an insulating layer (not shown in the Figure). The conductive wiring layers electrically connect the light-emitting unit 16 and the photosensitive unit 18, while the insulating layer severs to support the conductive wiring, the light-emitting unit 16, and the photosensitive unit 18. For example, the circuit substrate 12 can be a fiberglass substrate, a ceramic substrate, a flexible substrate, a metal substrate, a semiconductor substrate, and so on.

The isolating barriers 14 are formed on the circuit substrate 12. In this embodiment, three isolating barriers 14 are used as an example. The isolating barriers 14 form a first chamber 142 and a second chamber 144, which are partially enclosed, on the circuit substrate 12. The first chamber 142 is located on the first side of the second chamber 144, which is the right side in the Figure. The material of the isolating barriers is capable of blocking or absorbing light. In this embodiment, the width (also referred to as “thickness”) of each isolating barrier 14 is within the range of less than or equal to 0.3 cm, effectively achieving the goal of thinness. The material of the isolating barriers 14 can be black silicone, black epoxy resin, etc.

The light-emitting unit 16 is disposed on the circuit substrate 12 and situated in the first chamber 142. In this embodiment, two light-emitting units 16 are used as an example. In other embodiments, the number of the light-emitting units 16 is not limited to a specific number, and a single unit also falls within the scope of this invention. When the light-emitting unit 16 is activated, it emits a first light L1 having a wavelength within the wavelength band of the visible or non-visible light spectrum. For example, the first light L1 can be ultraviolet light, visible light, near-infrared, far-infrared, or any combination thereof. Depending on the number of light-emitting units 16, the same or different wavelengths may be employed. The first light L1 is directed towards human skin, and its ability to penetrate to the underlying skin tissue beneath the epidermis depends on its wavelength and power. Certain components within the tissue liquid alter the trajectory of the first light L1, generating reflected light which is referred to as the second light L2 hereinafter. That is, the second light L2 is the diffusely reflected light of the first light L1.

The photosensitive unit 18 is disosed on the circuit substrate 12 and situated in the second chamber 144. In this embodiment, one photosensitive unit 18 is used as an example, and in other embodiments, the number of photosensitive units 18 is not limited to a specific number. The photosensitive unit 18 can receive the second light L2 reflected from the epidermis. The sensing wavelength of the photosensitive unit 18 matches that of the light-emitting unit 16, and the photosensitive unit 18 is capable of detecting wavelengths across the visible and non-visible light spectrum.

The light-emitting unit 16 and photosensitive unit 18 can be attached to the circuit substrate 12 using wire bonding or “flip-chip bonding.”

The light-guiding assembly 20 is disposed between the isolating barriers 14 to enclose the first chamber 142 and the second chamber 144, such that the light-emitting unit 16 is enclosed in the first chamber 142, and the photosensitive unit 18 is enclosed in the second chamber 144. The light-guiding assembly 20 includes a first optical element 202 and a second optical element 204. The first light L1 generated by the light-emitting unit 16 is confined by the first chamber 142 so that the first light L1 can be transmitted out only through the first optical element 202.

A first transmitting layer 2022 is formed on one side of the first optical element 202 (herein, refers to the direction of the +Y axis in the Figure), and a first patterned layer 2024 is formed on another side of the first optical element 202 (herein, refers to the direction of the −Y axis in the Figure). A second transmitting layer 2042 is formed on one side of the second optical element 204 (herein, refers to the direction of the +Y axis in the Figure), and a second patterned layer 2044 is formed on another side of the second optical element 204 (herein, refers to the direction of the −Y axis in the Figure). The first transmitting layer 2022 (or the second transmitting layer 2042) can be formed on the surfaces of substrates such as optical glass, quartz, sapphire, and the like by coating processes. The first patterned layer 2024 (or the second patterned layer 2044) can be formed, with patterned concave-and-convex features, directly on the opposite surface of the substrate such as the optical glass mentioned above. Accordingly, transmitting layers and patterned layers are formed on the corresponding two sides of the optical glass. Depending on the material and thickness of the transmitting layer, it can determine the wavelengths that pass through and are cut off, achieving a filtering effect. Alternatively, by selecting the material and thickness of the transmitting layer, the transmitting layer can serve as an anti-reflective layer (AR) that alters the light transmittance, for example, increasing the light transmittance. One example of the material of the transmitting layer is magnesium fluoride (MgF2), and the thickness is equal to one-fourth of the wavelength of light. Herein, the first optical element 202 and the second optical element 204 are set parallel to the circuit substrate 12 for illustration, so that the first optical element 202 and the second optical element 204 are flat panels. The first light L1 can be transmitted outwards through the first optical element 202, and the second light L2 can be focused and directed inwards by the second optical element 204.

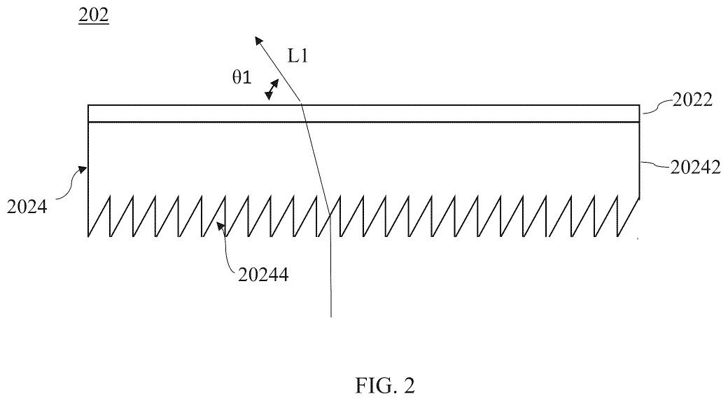

The first optical element 202 is disposed corresponding to the light-emitting unit 16, and the first patterned layer 2024 can alter the emitting angle θ1 of the first light L1. Refer to FIG. 2, which is an enlarged view schematically illustrating the first patterned layer according to the first embodiment of the present invention. As shown in FIG. 2, the first patterned layer 2024 has a plurality of first sawtooth structures 20242, each with a first inclined plane 20244. The inclined angle of the first inclined plane 20244 is less than or equal to 90 degrees, such as 45 degrees. The first inclined planes 20244 determine the emitting angle θ1 of the first light L1. For example, the first light L1 is emitted in a leftward oblique direction in the Figure, i.e., the first light L1 is directed toward the photosensitive unit 18, increasing the amount of light received by the photosensitive unit 18.

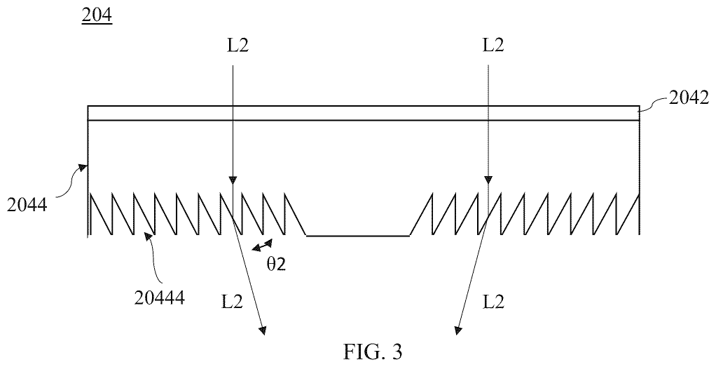

The second optical element 204 is disposed corresponding to the photosensitive unit 18, and the second patterned layer 2044 can alter the incident angle θ2 of the second light L2. Refer to FIG. 3, which is an enlarged view schematically showing the second patterned layer 2044 according to the first embodiment of the present invention. In FIG. 3, the second patterned layer 2044 has a plurality of second sawtooth structures 20442, each with a second inclined plane 20444. The inclined angle of the second inclined plane 20444 is less than or equal to 90 degrees, such as 45 degrees. Herein, the second patterned layer 2044 is featured in that the second sawtooth structures 20442 are symmetrically arranged with the center of the second optical element 204 as a symmetrical axis, so that the second inclined planes 20444 of the second sawtooth structure 20442 guide and concentrate the second light L2 onto the photosensitive unit 18. The second inclined planes 20444 determine the incident angle θ2 of the second light L2.

Therefore, the first light L1 is guided by the first optical element 202, such that the energy of the first light L1 is concentrated and directed to a predetermined region (not shown) on the epidermal layer. Herein, the predetermined region refers to an area biased towards the photosensitive unit, i.e. the area above the photosensitive unit. Additionally, the second light L2, transmitted from the predetermined region, is directed by the second optical element 204 and is consequently incident onto the photosensitive unit 18. Furthermore, the first light L1 and the second light L2 are blocked by the isolating barriers 14, ensuring that the first light L1 cannot be directly emitted to the photosensitive unit 18, and ensuring that the photosensitive unit 18 exclusively receives light reflected from the predetermined region.

It is noted that the biomedical detection photoelectric module 10 may further include a first glue 22 and a second glue 24. The first glue 22 encapsulates the light-emitting unit 16 in the first chamber 142, while the second glue 24 encapsulates the photosensitive unit 18 in the second chamber 144. The first glue 22 and the second glue 24 are made of transparent or semi-transparent materials, such as silicone gel, epoxy resin, and the like.

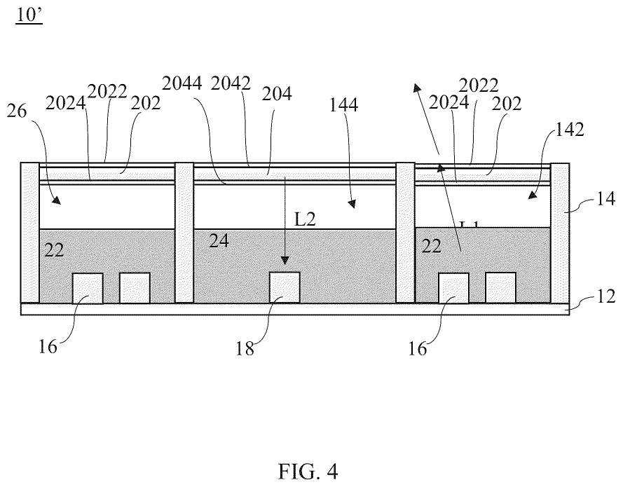

Refer to FIG. 4, which is a cross-sectional view schematically depicting the structure of the biomedical detection photoelectric module according to the second embodiment of the present invention. In FIG. 4, the biomedical detection photoelectric module 10′ includes a circuit substrate 12, a plurality of isolating barriers 14, light-emitting units 16, a photosensitive unit 18, and the light-guiding assembly 20, as described hereinbefore in connection with the first embodiment. In addition, the biomedical detection photoelectric module 10′ further includes a third chamber 26.

The circuit substrate 12, isolating barriers 14, light-emitting unit 16, photosensitive unit 18, and light-guiding assembly 20 may be the same as or similar to those described above and are not repeated here.

The third chamber 26 is created by the isolating barriers 14 on the circuit substrate. The third chamber 26 is located on the second side of the second chamber 144, where the second side corresponds to the first side. Further, the third chamber 26 and the first chamber 142 jointly encircle the second chamber 144. The third chamber 26 also has the light-emitting units 16 disposed therein, as described hereinbefore.

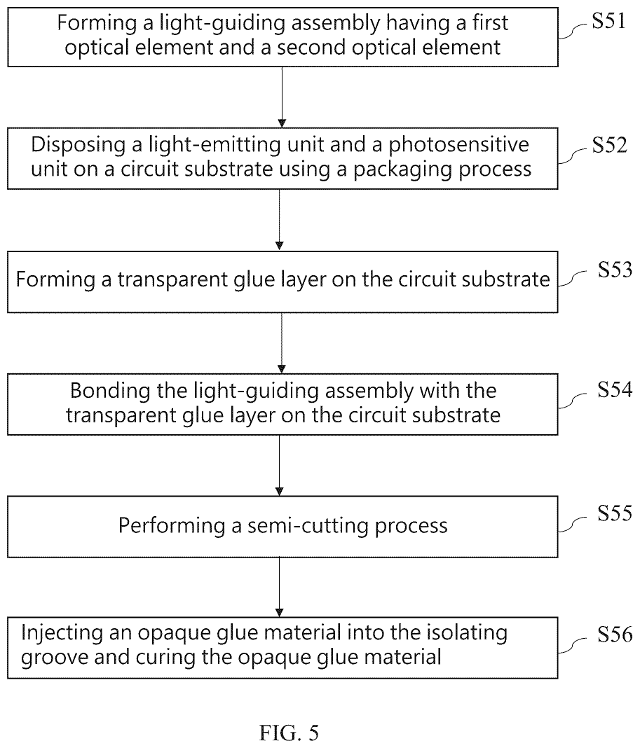

FIG. 5 is a flowchart illustrating a method for manufacturing a biomedical detection photoelectric module, in accordance with the third embodiment of the present invention. In FIG. 5, the method for manufacturing the biomedical detection photoelectric module begins at step S51, where a transmitting layer is formed on one side of a quartz substrate, and a first patterned layer and a second patterned layer are formed on another side of the quartz substrate to create a light-guiding assembly having a first optical element and a second optical element.

Next, in step S52, a light-emitting unit and a photosensitive unit are disposed on a circuit substrate through a packaging process.

Subsequently, in step S53, a transparent glue layer is formed on the circuit substrate to secure the light-emitting unit and the photosensitive unit to the circuit substrate.

Next, in step S54, a light-guiding assembly is bonded to the transparent glue layer on the circuit substrate.

Following that, in step S55, a semi-cutting process is performed to cut the light-guiding assembly and the transparent glue layer to form isolating grooves.

Subsequently, in step S56, an opaque glue material is injected into the isolating grooves and cured to separate the first chamber and the second chamber.

The foregoing has outlined features of several embodiments so that those skilled in the art may better understand the detailed description that follows. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A biomedical detection photoelectric module for detecting a state of tissue liquid beneath the epidermis, comprising:
a circuit substrate;
a plurality of isolating barriers formed on the circuit substrate, wherein the isolating barriers form a first chamber and a second chamber on the circuit substrate, with the first chamber being located on a first side of the second chamber;
a light-emitting unit disposed on the circuit substrate and situated in the first chamber, the light-emitting unit capable of emitting a first light;
a photosensitive unit disposed on the circuit substrate and situated in the second chamber, wherein the photosensitive unit is configured to receive a second light from the epidermis, wherein the second light is diffusely reflected light of the first light; and
a light-guiding assembly disposed between the isolating barriers to enclose the first chamber and the second chamber, wherein the light-guiding assembly comprises a first optical element and a second optical element, wherein the first optical element has a first transmitting layer formed on one side thereof and a first patterned layer formed on another side thereof, wherein the second optical element has a second transmitting layer formed on one side thereof and a second patterned layer formed on the other side thereof, wherein the first optical element is corresponding to the light-emitting unit, and the first patterned layer is configured to alter an emitting angle of the first light, wherein the second optical element is corresponding to the photosensitive unit, and the second patterned layer is configured to alter an incident angle of the second light;
wherein the first light is guided and concentrated onto a predetermined region of the epidermis by the first optical element, and the second light from the predetermined region is guided and concentrated onto the photosensitive unit by the second optical element, and wherein the first light and the second light are blocked by the isolating barriers, preventing direct incidence of the first light on the photosensitive unit.

2. The biomedical detection photoelectric module of claim 1, wherein the circuit substrate has conductive wiring and an insulating layers, wherein the conductive wiring electrically connects the light-emitting unit and the photosensitive unit, and the insulating layer supports the conductive wiring, the light-emitting unit, and the photosensitive unit.

3. The biomedical detection photoelectric module of claim 1, wherein the first optical element and the second optical element are parallel to the circuit substrate, allowing the first light to pass through the first optical element and the second light to pass through the second optical element.

4. The biomedical detection photoelectric module of claim 1, wherein the first patterned layer has a plurality of first sawtooth structures, wherein each of the first sawtooth structures has a first inclined plane, and the first inclined planes determine the emitting angle of the first light.

5. The biomedical detection photoelectric module of claim 1, wherein the second patterned layer has a plurality of second sawtooth structures, wherein each of the second sawtooth structures has a second inclined plane, and the second inclined planes determine the incident angle of the second light.

6. The biomedical detection photoelectric module of claim 5, wherein the second patterned layer is characterized in that the second sawtooth structures are symmetrically arranged with a center of the second optical element as an axis of symmetry, such that the second inclined planes of the second sawtooth structures direct and concentrate the second light onto the photosensitive unit.

7. The biomedical detection photoelectric module of claim 1, wherein an amount of each of the light-emitting unit and the photosensitive unit is single or plural.

8. The biomedical detection photoelectric module of claim 1, where the isolating barriers form a third chamber on the circuit substrate, and the third chamber is situated on a second side of the second chamber, wherein the second side is located on a corresponding side to the first side, such that the third chamber and the first chamber surround the second chamber, and a light-emitting unit is disposed in the third chamber.

9. The biomedical detection photoelectric module of claim 1, where surfaces of the first optical element and the second optical element are substantially flat.

10. The biomedical detection photoelectric module of claim 1, further comprising a first glue and a second glue, wherein the first glue covers the light-emitting unit in the first chamber, and the second glue covers the photosensitive unit in the second chamber.

11. The biomedical detection photoelectric module of claim 10, where the first glue and the second glue are made of a transparent or semi-transparent material.

12. The biomedical detection photoelectric module of claim 1, where the first transmitting layer and the second transmitting layer are provided for a specific wavelength band to pass through.

13. The biomedical detection photoelectric module of claim 1, where each of the first transmitting layer and the second transmitting layer is an anti-reflective layer to alter a light transmittance.

14. A method for manufacturing a biomedical detection photoelectric module, the method comprising:
forming a transmitting layer on one side of a quartz substrate and forming a first patterned layer and a second patterned layer on another side of the quartz substrate to form a light-guiding assembly having a first optical element and a second optical element;
disposing a light-emitting unit and a photosensitive unit on a circuit substrate using a packaging process;
forming a transparent glue layer on the circuit substrate to secure the light-emitting unit and the photosensitive unit on the circuit substrate;
bonding the light-guiding assembly with the transparent glue layer on the circuit substrate;
performing a semi-cutting process to cut the light-guiding assembly and the transparent glue layer to form at least one isolating groove; and
injecting an opaque glue material into the isolating groove and curing the opaque glue material to separate a first chamber from a second chamber.
